(19) United States
(12) Patent Application Publication
(10) Pub. No.: US 2026/0207692 A1
(43) Pub. Date: Jul. 23, 2026

(54) PHARMACEUTICAL COMPOSITION FOR TREATING A BRAIN TUMOR

(71) Applicant: PATENTPOOL TARGET GMBH, Muenchen (DE)

(72) Inventors: Renate BURDA, Sauerlach (DE); Jenny MÖRTH-KRETSCHMER, Buchholz in der Nordheide (DE); Elisabeth SAND, Rauenzell/Herrieden (DE); Dirk WEICKMANN, Rauenzell/Herrieden (DE)

(73) Assignee: PATENTPOOL TARGET GMBH, Muenchen (DE)

(21) Appl. No.: 19/142,286

(22) PCT Filed: Nov. 23, 2023
(86) PCT No.: PCT/EP2023/082810

§ 371 Date: Jun. 23, 2025

(30) Foreign Application Priority Data
Dec. 24, 2022 (EP) 22216672.0

(51) Int. Cl. A61K 36/185; A61K 35/583; A61K 35/76; A61K 38/16; A61P 35/00

(52) U.S. Cl. CPC A61K 36/185; A61K 35/583; A61K 35/76; A61K 38/168; A61P 35/00; A61K 2236/39; A61K 2236/53; C12N 2710/24232

(57) ABSTRACT

The invention relates to a pharmaceutical composition for the treatment of a brain tumor, comprising in a pharmaceutically effective amount peptide toxin from plants of the genera Dendrocnide and/or Urera and/or Nanocnide and/or Girardinia; and total toxin or parts of the toxin from plants of the genus Laportea as well as the use of the pharmaceutical composition for the treatment of a brain tumor, a process for the preparation of the pharmaceutical composition and a kit of parts comprising the pharmaceutical composition.

The invention relates to a pharmaceutical composition for the treatment of a brain tumor, comprising in a pharmaceutically effective amount peptide toxin from plants of the genera Dendrocnide, Urera, Nanocnide and/or Girardinia; and total toxin or parts of the toxin from plants of the genus Laportea as well as the use of the pharmaceutical composition for the treatment of a brain tumor, a process for the preparation of the pharmaceutical composition and a kit of parts comprising the pharmaceutical composition.
The present invention relates to a pharmaceutical composition, the use of the pharmaceutical composition for treating a brain tumor, a process for preparing the pharmaceutical composition and a kit of parts containing the pharmaceutical composition.
In Germany, around 9,000 cases of malignant brain tumors occur every year. These are astrocytomas with grade II and III malignancy, as well as grade IV glioblastomas and glioblastoma multiforme. Even today, a brain tumor is usually diagnosed as an incidental finding. However, bleeding in the skull and brain area of patients also occurs regularly, which is accompanied by unconsciousness and/or severe, sometimes very severe pain. This makes hospitalization unavoidable.
The treatment of brain tumors is extremely difficult. Especially in the case of glioblastomas, the prognosis for the patient is usually not positive. Surgical removal of the tumor can extend the survival time by a few months and alleviate the symptoms. However, a permanent cure is extremely rare and almost impossible in the case of glioblastomas from a conventional medical point of view.
Tumors are currently regarded as the most dangerous and most feared diseases of our time. They are fought in a very radical way that is not very gentle on the patient. Simple key words that characterize them are:
Steel, radiation and chemotherapy.
This means that tumors, if reasonably accessible, are in principle surgically excised with a scalpel, destroyed by broad-spectrum radiation, or destroyed by chemotherapy with aggressive chemotherapeutic/cytostatic agents that also attack healthy cells. In both normal treatments with a scalpel and with ionizing radiation, it is not possible to limit the surgical area. Healthy body cells are also inevitably destroyed. The undesirable side effects of chemotherapy are generally known, as are the disadvantages of radiotherapy.
Vaccines are not available and from a cell biological and biochemical point of view are rather counterproductive as far as the use in diseases with cell degeneration is concerned.
However, the surgical removal of brain tumors in particular has other side effects. The patient often requires a second operation. During the first operation and removal of the brain tumor, a large scar area is created. In addition to healthy glial cells, diseased, i.e. malignant, glial cells can also accumulate there. These are often cells that originate from the tumor and produce metastases. This means that a diffuse recurrence is pre-programmed. This can no longer be treated using conventional treatment methods. It is therefore often fatal for a patient.
Surgery is usually followed by radiotherapy and/or chemotherapy. For chemotherapy, cisplatin is often used in various combinations. Since the beginning of 2002, oral temozolomide has also been used. IMATINIB is also an orally administered cytostatic drug that is used. In addition to the known side effects such as vomiting, malaise, depression, cancer caused by chemotherapy and nervous disorders, the problem with the above-mentioned drugs is that they have to cross the blood-brain barrier to treat the brain tumor. The active ingredients usually only cross the blood-brain barrier by 0.4 to 5%. The composition and function of the blood-brain barrier are still only partially understood. This barrier plays a role in protecting the brain from harmful substances, but also enables a regulated supply of energy.
Our body consists of individual organ systems and organs that require different but constant conditions for their function, for example nutrients, hormones or electrolytes. All organs are connected to each other via the blood circulation. As the blood contains all the components needed to supply and purify the body, filter systems must ensure that only the substances required by the individual organ systems are allowed to pass through or are partially retained. The blood-tissue barrier, also known as the blood-parenchyma barrier, the blood-liver barrier, the blood-cerebrospinal fluid barrier, the blood-brain barrier, the cerebrospinal fluid-brain barrier, the blood-nerve barrier, the blood-retina barrier and the placenta barrier are known in this context.
These filter mechanisms use a so-called barrier effect to prevent or restrict the transfer of certain substances from the bloodstream into the respective organ system if these organ systems do not require the components or only require them in lower concentrations.
These “barriers” are not independent organs, but are formed from a large number of cells and intercellular spaces that allow blood gases, nutrients and certain chemicals to pass through or, as endothelial pores, retain macromolecules. As lipid membranes in the vascular wall, they can also inhibit the passage of non-lipid-soluble substances or have a selective effect on active transport processes in the capillaries. The brain and nervous tissue are protected by two filter systems, the blood-cerebrospinal fluid barrier and the blood-brain barrier.
The existence and function of the so-called blood-brain barrier has been known for over 100 years and was demonstrated experimentally by Paul Ehrlich as early as 1885. Within the central nervous system, the spaces between the neurons are almost completely filled by glial cells and their extensions. The entire metabolism of the nerve cells takes place via these glial or endothelial cells. They serve to incorporate the nerve cells and nerve fibers and to nourish and insulate them. Astrocytes are a type of glial cell. They have numerous projections with which they attach themselves to the wall of the capillaries and form a virtually gapless endothelial lining that surrounds the capillaries on all sides. These endothelial cells are linked by connecting elements, the “tight junctions”, and are equipped with a selective substance permeability that only allows particles with a diameter of less than 20 nm to pass through. In this way, the entire metabolism of the nerve cells passes through this endothelial plexus, which allows the substances present in the blood to pass through as required like a biological filter, but keeps substances harmful to brain function away from the nervous system.
This endothelial plexus and the endothelial cells that line the capillaries as a basement membrane are known as the blood-brain barrier. Oxygen, carbon dioxide, D-glucose, D-hexose, some L-amino acids and lipid-soluble substances, which are necessary for supplying the brain, pass through unhindered. Degradation products are also released into the blood. The terminal processes of the astrocytes represent a certain barrier for numerous substances such as certain hormones, non-lipid-soluble, water-soluble and chemical substances as well as proteins, thereby ensuring the maintenance of a constant environment for the neurons of the nervous system.
The cell structure of astrocytes is arranged in such a way that it forms an effective barrier against higher molecular weight substances and organisms. However, even under normal conditions it is not completely impermeable, so that some particles can always penetrate this barrier. In the case of infections, trauma, inflammation, poisoning, hypoxia, fever and in the area of tumors, the tight junctions between the endothelial cells are stretched by the swelling of the astrocytes and become significantly more permeable to other substances. The change in light transmission occurs through swelling and deswelling of the endothelial cells. The basement membrane of the capillaries is also not a closed layer. Depending on the density of the fiber network, pores are formed in the membrane that are actively involved in the exchange of substances.
Long before the possibility of antibiotic treatment, the permeability of the blood-brain barrier was increased by artificially creating a fever similar to the process in infections and used to treat syphilis of the central nervous system and for shock treatment in psychiatry by delivering drugs directly to the brain. Once the conditions influencing the blood-brain barrier have ceased to have an effect, the temporary permeability is restored.
Termozolomide, a lipophilic alkylan, is currently being clinically researched for the chemotherapy of patients with brain metastases of solid tumors. It crosses the blood-brain barrier and increases the radiosensitivity of tumors during simultaneous radiotherapy.
In contrast to this, however, attempts have also been made to enable cancer therapy in a more subtle way based on natural substances.
Among other things, many highly effective substances isolated from poisonous organisms are used in therapeutic doses as medicinal substances.
The use of these biogenic poisons began early in the history of mankind. However, in order to use these poisons safely, certain basic knowledge about their treatment and effectiveness was required from the very beginning. Further attempts to decipher the composition of the chemical structure of biogenic toxins later led to the targeted search for specific active substances as the actual cause of observed effects.
The development of chromatographic processes in the middle of the 20th century made a huge leap forward possible in separation technology, the way to identify active substances to combat diseases. Based on the distribution between a mobile and a stationary liquid phase, adsorption, molecular sieve effects, ion exchange, affinity (especially of proteins) to certain chemical compounds (e.g. enzyme substrates) and the mobility of charged molecules in an electric field, a large number of new separation techniques were developed.
In recent times in particular, many active pharmaceutical ingredients have been isolated and further developed from biogenic toxins (from fungi, bacteria, plants and animals).
For example, PCT/EP00/12902 discloses a pharmaceutical active ingredient for which it was found that components of the spider venom of spiders of the Sicariidae family can be used to treat tumor diseases. A peptide toxin from the venom of this spider species, a further antagonist substance obtained from the venom and/or a combination of these components are mainly used medically. The use of peptide toxins from the genus Sicarius, for example, is proposed for the treatment of mammary carcinomas, lung carcinomas, adenocarcinomas, liver carcinomas and melanomas. The treatability of brain tumors such as astrocytomas and glioblastomas, on the other hand, is not mentioned. Consequently, the problem of overcoming the blood-brain barrier is also not discussed.
The active substances described in PCT/EP00/12902 can be used to treat tumor diseases as well as in parallel or in support of tumor operations and to destroy residual tumor tissue. Genetically modified body cells (tumor cells) can be destroyed during therapy, as the active substance in question recognizes the modified surface structure of such cells and kills them without complications. The total venom of these spider species, a cocktail of different substances so to speak, cannot be used pharmaceutically due to its lethal effect even at low doses.
However, this known active substance does not work in vivo in any combination with a brain tumor, especially not with a particular type of brain tumor, namely an oligodendroglioma or oligodendrocytoma. In addition, it is necessary for this active substance to cross the blood-brain barrier to a large extent for successful therapy.
A further combination of toxins is disclosed in PCT/EP2006/063281. However, the combination of different toxins disclosed therein partially neutralizes each other, so that the desired tumor cell-destroying effect is only insufficiently achieved.
Surprisingly, it turned out that the peptide toxins of Loxosceles degrade the peptide toxins of Latrodectus, so that there is no significant penetration of the blood-brain barrier by the Loxosceles peptide toxins. After mixing the peptide toxins of Latrodectus and Loxosceles, no distinct bands appeared on an electrophoresis gel, but only a smear caused by the fragments of the peptide digest of the Latrodectus peptide toxins. Spiders of the genus Loxosceles, like spiders of the genus Sicarius, belong to the family Sicariidae. Due to the close relationship between the two spider genera, a mixture of peptide toxins from the genus Sicarius with those from the genus Latrodectus was therefore not expected to have any effect.
It is therefore a task of the present invention to provide a composition which, while overcoming the blood-brain barrier as effectively as possible, causes the uncomplicated destruction of cancerous body cells from the area of brain tissue, in particular the rare brain tumor oligodendroglioma.
Another object of the present invention is to provide a process which enables the preparation of a pharmaceutical composition for the treatment of brain tumors.
It is a further task of the present invention to provide a combination of active ingredients that enables effective treatment of brain tumors.
These tasks are solved by the features of the independent claims. Preferred further developments and embodiments of the invention can be found in the sub-claims.
Thus, a first aspect of the present invention relates to a pharmaceutical composition for treating a brain tumor comprising, in a pharmaceutically effective amount
1.(a) peptide toxin from plants of the genus Dendrocnide and/or plants of the genus Urera and/or plants of the genus Nanocnide and/or plants of the genus Girardinia; and
2.(b) containing peptide toxin from plants of the genus Laportea

The peptide toxin of plants of the genus Dendrocnide and/or plants of the genus Urera and/or plants of the genus Nanocnide and/or plants of the genus Girardinia is able to destroy cells of a tumor. When combined with the poison of plants of the genus Laportea, the tumor-destroying poison is transported past the blood-brain barrier into the brain, where it can fight the brain tumor. The poison of the plants of the genus Laportea thus acts as a penetrating substance for the blood-brain barrier. Only this special combination makes it possible to destroy tumor tissue in the brain. Unexpectedly, the peptide toxins of Laportea were not degraded by the peptide toxins of Dendrocnide and/or Urera and/or Nanocnide and/or Girardinia, so that the Laportea peptide toxins cross the blood-brain barrier as transfer substances for the other tumor cell-destroying toxins and, in combination with the peptide toxins of Dendrocnide and/or Urera and/or Nanocnide and/or Girardinia, were able to destroy the tumors in the brain area, for example oligodendrocytes.

The peptide toxins can be obtained in a known manner. Full reference is made to the description of PCT/EP00/12902. The respective total toxin mixtures can be separated into individual fractions by various chromatographic methods, for example by HPLC techniques, and the fractions can then be examined for their efficacy. The tumor cell-destroying effect of individual peptide toxin fractions can be determined in cell culture experiments. The efficacy of the tumor cell-destroying peptide toxin fractions in combination with the blood-brain barrier penetrating substance from Laportea is then determined in known animal models and cell experiments. Examples thereof are given in PCT/EP00/12902, which is fully referenced herein and the contents of which are incorporated in their entirety in the present application. As described in PCT/EP00/12902 with reference to Sicarius peptide toxins, the skilled person can also isolate the active constituents for the peptide toxins from the plant genera Dendrocnide, Urera, Nanocnide and Girardinia and from the total toxin cocktail7 extract of plants of the genus Laportea and test them as described above. Reference is also made to PCT/EP2006/063281, the content of which is also incorporated in its entirety in the present application and to which the applicant refers here. This application shows the use of peptide toxins from Laportea, which are able to penetrate the blood-brain barrier and at the same time have the ability to allow other peptide toxins, namely those from Dendrocnide, Urera, Nanocnide, Girardinia, to penetrate the blood-brain barrier.

In Laportea, as an alternative to the peptide toxin fractions, the total toxin/total toxin extract of the plants can also be used. Preferably, only those fractions from the total toxin cocktail/total toxin extract of Laportea are used in the pharmaceutical composition which contain the active ingredients which are capable of passing the blood-brain barrier or which enable the antitumor active ingredients from dendrocnide and/or urera and/or nanocnide and/or girardinia to overcome this barrier.

According to a preferred further development of the present invention, the pharmaceutical composition additionally contains inactivated Parapoxvirus ovis, preferably of strain D1701 This is inactivated Parapoxvirus ovis of strain D1701, to which polygelins and aqua ad injectionem have additionally been added.

According to a further preferred embodiment of the present invention, the plants of the genus Dendrocnide are selected from the group consisting of the species Dendrocnide corallodesme and/or Dendrocnide cordata and/or Dendrocnide cordifolia and/or Dendrocnide excelsa and/or Dendrocnide gigantea and/or Dendrocnide meyeniana and/or Dendrocnide moroides and/or Dendrocnide peltata and/or Dendrocnide sinuata and the plants of the genus Urera selected from the group consisting of the species Urera caracasana and/or Urera baccifera and/or Urera expansa and/or Urera kaalae Urera nitida and/or Urera simplex and the plants of the genus Nanocnide selected from the group consisting of the species Nanocnide japonica and/or Nanocnide lobata and/or Nanocnide closii and the plants of the genus Girardinia selected from the group consisting of the species Girardinia bullosa and/or Girardinia diversifolia.

The plants of the genera Dendrocnide and/or Urera and/or Nanocnide and/or Girardinia and/or Laportea are preferably extracted as follows in order to obtain the total poison cocktail of the plants:

The leaves of the plants are harvested at temperatures of 24 to 31 degrees Celsius. The leaves are extracted with common peptide solvents. The resulting total toxin cocktail is broken down into its individual components using a column chromatography and/or other conventional methods.

In Example 2, the extraction of dendrocnide peptide toxin, urera peptide toxin, nanocnide peptide toxin and girardinia peptide toxin is described in more detail. The details for obtaining peptide toxins from dendrocnide, urera, nanocnide, girardinia, which are also applicable to obtaining peptide toxins from Laportea, are given in PCT/EP00/12902, so that the skilled person can easily obtain the effective fractions of the plant (Urticaceae) toxins using this prior art.

According to another preferred further development of the present invention, the peptide toxin from plants of the genus Dendrocnide is the HTDen1 and/or the HTDen2 peptide toxin. Example 2 describes the preparation of these peptide toxins in more detail.

According to another preferred further development of the present invention, the composition additionally contains Lachesis D6. The Lachesis D6 (Lachesis means pit viper) of the DHU (German Homeopathic Union) is preferably used as a solvent for the dendrocnide peptide toxin and/or the urera peptide toxin and/or the nanocnide peptide toxin and/or the girardinia peptide toxin. In particular, Lachesis D6 can be advantageously used in the preparation of the composition.

According to another preferred embodiment of the present invention, the plants of the genus Laportea are selected from the group consisting of the species Laportea bulbifera, Laportea canadensis, Laportea cuspidata, Laportea grossa, Laportea interrupta, Laportea mooreana, Laportea ruderalis, Laportea septentrionalis.

According to a particularly preferred further development of the present invention, the plants of the genus Laportea are preferably the species Laportea bulbifera, Laportea canadensis and Laportea ruderalis.

Mixtures of different poisons of the genera Dendrocnide or Urera or Nanocnide or Girardinia can also be made according to another further development of the present invention. The mixture may concern different species of the genus Dendrocnidae or Urera or Nanocnidae or Girardinia or Laportea or also mixtures of different populations of one species.

A second aspect of the present invention relates to the use of a pharmaceutical composition as described above for the manufacture of a medicament for the treatment of brain tumors and glioblastomas, in particular for the treatment of an oligodendroglioma.

The combination of venom or parts of the venom of plants of the genus Dendrocnide or Urera or Nanocnide or Girardinia with a venom or parts of the venom of plants of the genus Laportea according to the invention overcomes the blood-brain barrier. The venom of the Laportea plants serves as a penetrating substance that enables the barrier to be penetrated.

Another aspect of the present invention relates to a method of preparing a composition comprising the steps of:

Preparation of an isotonic (0.9%) sodium chloride solution with dendrocnide peptide toxin and/or urera peptide toxin and/or nanocnide peptide toxin and/or girardinia peptide toxin
optionally adding a further dendrocnide peptide toxin and/or urera peptide toxin and/or nanocnide peptide toxin and/or girardinia peptide toxin to the sodium chloride solution; and
Addition of a total toxin or peptide toxins from plants of the genus Laportea with a molecular weight of 5 kDa to 130 kDa and the ability to penetrate the blood-brain barrier;
Mixing the ingredients, in particular by shaking the mixture.

According to a preferred further development of this aspect, an immunomodulator is additionally added before mixing, preferably Parapoxvirus ovis, in particular preferably of strain D1701.

According to another preferred further development of this aspect, at least one of the dendrocnide peptide toxins and/or urera peptide toxins and/or nanocnide peptide toxins and/or girardinia peptide toxins is dissolved in Lachesis D6 before it is added to the NaCl solution, preferably both sicarius peptide toxins.

According to another preferred further development of this aspect, the first dendrocnide peptide toxin is a dendrocnide peptide toxin HTDen1 and/or the second dendrocnide peptide toxin is a dendrocnide peptide toxin HTDen2.

Furthermore, peptide toxins from dendrocnide species are preferably used as penetrating substances in the pharmaceutical preparation according to the invention, which facilitate the uptake of the peptide toxins into the tumor cell. Such substances and their extraction are described, for example, in PCT/EP00/12902.

A further aspect of the present invention relates to a kit of parts for the treatment of brain tumors and glioblastomas, comprising a pharmaceutical composition as described above and an extract of frankincense and/or bamboo. Particularly preferred herein is the extract of frankincense and/or bamboo leaves.

The main components of frankincense are resins, which contain boswellic acids and essential oils. In addition to the boswellic acids, which belong to the group of pentacyclic triterpenes, a tetracyclic triterpene, tirucallic acid, is also found in the resins.

Preferably, the preparation H15 Ayurmedica is used in the present invention. These are tablets, each containing 400 mg of standardized dry extract of Boswellia serata. The dosage is adjusted according to need. It can vary widely and is usually in the range of 3 tablets per week up to 25 tablets per day, preferably 10 tablets per week up to 20 tablets per day.

The frankincense extract is used as an alternative to cortisone to prevent edema in the brain during treatment with the pharmaceutical composition according to the present invention. Edema occurs more frequently in the treatment of brain tumors, especially when they are rapidly destroyed, i.e., when they rapidly decrease in size.

The various toxins are explained in detail below.

The tumor cells are destroyed by peptide toxins, which can be obtained from the venom of plants of the genera Dendrocnide and/or Urera and/or Nanocnide and/or Girardinia. Preference is given to the species Dendrocnide corallodesme, Dendrocnide cordata, Dendrocnide cordifolia, Dendrocnide excelsa, Dendrocnide gigantea, Dendrocnide meyeniana, Dendrocnide moroides, Dendrocnide peltata, Dendrocnide sinuata, Urera caracasana, Urera baccifera, Urera expansa, Urera kaalae, Urera nitida, Urera simplex, Nanocnide japonica, Nanocnide lobata, Nanocnide closii, Girardinia bullosa, Girardinia diversifolia.

The peptide toxins can be obtained by known separation methods, such as gel electrophoresis or chromatography, in particular column chromatography. The extraction of the crude toxin cocktail/extract from the plants is described below.

To cross the blood-brain barrier, active ingredients are preferably obtained from the venom of plants of the genus Laportea. These belong to the nettle family (Urticaceae). They can be found in a wide variety of areas, some of which are also found in cities. Many species prefer forests as their habitat.

The following types are preferred:
1.a) Laportea bulbifera
2.b) Laportea canadensis
3.c) Laportea cuspidata
4.d) Laportea grossa
5.e) Laportea interrupta
6.f) Laportea mooreana
7.g) Laportea ruderalis
8.h) Laportea septentrionalis

The two species Laportea bulbifera and/or Laportea ruderalis are particularly preferred for obtaining the peptide substances to cross the blood-brain barrier when treating children.

Particularly preferred in the composition according to the present invention are the species Laportea bulbifera and Laportea grossa.

Laportea (Laportea canadensis), as it is used in homeopathy, is mainly found in America along the Pacific coast as far as Canada.

Laportea bulbifera is found in Sri Lanka, India, Bhutan, Myanmar, Thailand, Vietnam, China, Korea, Russia, Japan and Indonesia.

Latrodectus cuspidata occurs in China, Japan, Korea and Myanmar.

Laportea grossa is found in R.S.A.

Laportea interrupta is found in India, Sri Lanka, Indonesia, Myanmar, Thailand, Malaysia, Vietnam, Yunnan, Taiwan and Australia

Laportea mooreana

Laportea ruderalis is known from the Solomon Islands, Christmas Island, Micronesia, French Polynesia, Samoa, Tonga and the Cook Islands

Laportea septentrionalis

The Laportea venom consists of 5 to 14 different proteins with molecular weights of 15 kDa to 100 kDa. The venom cocktail or parts of the venom cocktail of the Laportea species mentioned under a) to h) and in particular the species Laportea bulbifera and Laportea grossa are used. The Laportea species mentioned under a) and g) can also be used on children up to the age of about 12 years.

The substances used for the pharmaceutical composition according to the invention can be obtained naturally from the plants. These toxins were originally developed for defense against predators. This natural mode of action can be preserved by a function-preserving, gentle extraction of the basic poisonous substance (e.g. by manual harvesting of the leaves).

According to one embodiment of the invention, manual leaf extraction from the plants is provided. In this way, genuine, unadulterated native poisons are obtained. By default, analysis and/or quality control of the crude toxin mixture can be performed by electrophoretic methods.

The room temperature is usually around 25 to 31 degrees, the humidity 50% to 90%. The time of day is irrelevant.

It is also possible to produce the active substances described, which are contained in the toxins of the various plants, chemically synthetically or by genetic engineering methods in recombined form. As is usual with chemical substances, the present invention also comprises derivatives and salts of the substances provided according to the invention. For example, the peptide toxin may comprise one or more substitutions and/or deletions of amino acids, whereby it must of course be ensured that the medical effect according to the invention is retained. The active ingredient described is obtained by methods customary in chemical process engineering.

The active ingredients of the present invention are preferably used in the form of such a pharmaceutical composition in which they are mixed with suitable carriers or excipients in doses such that the condition is treated or at least alleviated. Such a composition may contain (in addition to the active ingredients and carrier) diluents, bulking agents, salts, buffers, stabilizers, solubilizing agents and other materials well known in the art. The term “pharmaceutically acceptable” is defined as a non-toxic material that does not interfere with the effectiveness of the biological activity of the active ingredient or active substance. The choice of carrier depends on the route of administration.

The pharmaceutical composition may additionally contain other agents that enhance the activity of the active ingredient or complement its activity or use in the treatment. Such additional factors and/or agents may be included in the pharmaceutical composition to achieve a synergistic effect or to minimize side effects or undesirable effects.

Techniques for formulating or preparing and administering the compounds of the present application can be found in “Remington's Pharmaceutical Sciences”, Mack Publishing Co, Easton, PA, latest edition. A therapeutically effective dose further refers to an amount of the compound sufficient to provide symptomatic improvement, such as treatment, cure, prevention or amelioration of such conditions. Suitable routes of administration may include, for example, oral, rectal, transmucosal or intestinal administration and parenteral administration, including intramuscular, subcutaneous, intramedullary injections as well as intrathecal, direct intraventricular, intravenous, intraperitoneal or intranasal injections. Subcutaneous or oral administration to a patient is preferred.

Administer 1-5 ml, preferably 2-4 ml of the composition at intervals of about 1-4 days. However, the amount of composition administered can be adjusted accordingly by a specialist. The adjustment can also be made during treatment.

With conventional treatment methods, both primary tumors and secondary or recurrent tumors tend to form edema, which causes increased pressure pain. Cortisone, often in very high doses, is frequently used to counteract the formation of edema. However, cortisones can also trigger tumors. In addition, cortisone can be highly addictive in its mode of action, so that it usually needs to be slowly phased out in many reducing partial doses.

The inventors of the present invention have also found that the compositions according to the invention are interfered with by cortisones in their passage into the brain. Surprisingly, however, the inventors have found that frankincense and/or bamboo extract can be used as a fully effective substitute against edema formation.

As already mentioned above, H15 preparations are the preferred incense extract. Bamboo extract, in particular the extract of bamboo leaves, may be considered. However, directly dried frankincense leaves and/or bamboo leaves in capsule form can also be administered in combination with the pharmaceutical composition according to the present invention.

In the following, the preparation and the mode of action of the pharmaceutical composition according to the present invention are illustrated by means of examples. These examples are merely exemplary and are not intended to limit the invention in any way.

To prepare a pharmaceutical composition in the form of a solution for injection or oral administration, first add 1 ml of a saturated solution of dendrocnide peptide toxin HTDen1 in Lachesis D6 DHU (German Homeopathic Union) to 30 ml of a 0.9% NaCl solution. Subsequently, 1 ml of a saturated dendrocnide peptide toxin HTDen2 solution is added to Lachesis D6 DHU. Both steps are preferably carried out at room temperature.

This mixed cocktail is completed by adding a substance from Laportea bulbifera or Laportea septentrionalis. The toxins of different populations of Laportea spp. can also be used.

The resulting mixture is then shaken 10 times towards the center of the earth.

The resulting composition can be stored at about 7° C. for about 12 months in the absence of light. The experimentally determined (electrophoretic) loss of efficacy after 12 months is about 4%.

In a cell culture flask (1250 ml), the efficacy of the different venom extracts is determined by counting the cells after 24, 48 and 100 hours. It can be seen that the cell population decreases only insignificantly. A decrease in the cell population in vitro corresponds to a reduction in the tumor in vivo.

It can be seen that hardly any cell growth takes place after increasing incubation of the cells. As a rule, the dendrocnide toxin, the urera toxin, the nanocnide toxin and the girardinia toxin progressively kill the tumor cells. A peptide toxin from Dendrocnide is used as the penetrating enzyme. This penetrating substance is available in sufficient quantities and facilitates the diffusion of peptide toxins such as dendrocnide toxin into the tissue and cells.

### THERAPY EXAMPLES

For the therapy of brain tumors, patients are administered a subcutaneous injection or an oral solution of 2-4 ml of a composition according to the invention every two or three days, depending on the severity of the disease. The course of therapy is monitored by positron emission tomography (PET) and via the tumor marker LSA (lipid-bound sialic acid). The therapy can be adjusted accordingly if necessary.

In addition, up to 20 tablets of H15 or 3 capsules of frankincense leaves are taken daily to prevent the formation of edema, which can also occur if the tumor is destroyed too quickly.

The capsules containing frankincense leaves also preferably contain dry extracts of Bambusa ventricosa.

Course of treatment based on various patient examples
Patient, born in 1997, glioblastoma diagnosed at the age of 5. Inoperable, received only 3 radiation treatments until July 2004. In August 2004 start of therapy with the composition according to the present invention, initially 2 ml daily for 5 days, then 2.5 ml every 2nd day until August 2008, from September 2008 every 3 days. From 2004 September the girl went back to kindergarten, then to school. Now a young woman, she is working and feels well. The LSA “started” at 27 and has leveled off at around 21.4 since November 2005. The woman takes 2 incense capsules once a week to prevent severe edema. The PET has been normal since 2009. The last recorded severe headaches were in July 2012.
Patient, born 1968, grade III astrocytoma, diagnosed in April 2016. Came to a Freiburg doctor in June 2016, after his 1st operation. Received 9 radiotherapy sessions after the operation and started temozolomide chemotherapy, which he tolerated poorly. By January 2017, the computer tomogram showed a strong diffuse spread of the tumor and a cell type of glioblastoma. In May 2017, the patient wanted to start nettle toxin therapy. He initially started with a daily dose of 5 mL from May 15-22, 2017 and then took 4 mL orally 3 times a day until Oct. 15, 2017 (patient is afraid of injections). From October 17 until now, 2.5 mL every 2 days. The patient's general condition is good and no more tumor detection is currently possible in the PET. The LSA was around 28 after the operation, 25 at the end of October 2017 and has stabilized at around 21 since 2019. The patient has been taking 5 tablets of H15 twice a day since May 2017.

### Example 2: General Representation of a Column Chromatographic Separation Process

For the separation of the effective peptide toxin fractions from the total toxin cocktails of plants of the genera Dendrocnide, Urera, Nanocnide and Girardinia and Laportea, column chromatographic separation methods can preferably be used. The following is a general description of a column chromatographic separation process that can be used according to the invention. Of course, a person skilled in the art can also use other methods for separating peptide mixtures. The individual fractions contained by the separation process are tested for their ability to destroy tumor cells, either on cell cultures or in animal experiments. The usability of individual fractions from the Laportea total toxin cocktail as a blood-brain barrier-crossing agent is preferably tested in animal models and in modified cell experiments.

### General Description of a Column Chromatographic Separation Process:

For separation into the individual components/fractions, the total toxin is taken up in 5 mL protein solvent for column chromatography, which is composed of 0.25 M Tris/HCl, pH 6.5 to 7.3, 1.92 M glycine in distilled and deionized water. A saturated solution is prepared with the total toxin cocktail in a Teflon vial. For homogeneous mixing of the total toxin cocktail with the protein solvent, shake on the vortex for 60 seconds while avoiding foam formation.

After homogenization, the solution is poured through a Plexiglas funnel into a transparent and upright Plexiglas column, which has an inner diameter of 1 cm, a wall thickness of 2 mm and a height of 50 cm and is conical at the bottom up to approx. 1.5 mm, but is open. The column contains 15 mL gel (ACA 34; matrix 3% acrylamide 4% agarose; fractionation range (MW): proteins from 20-350 kDa, exclusion limit: 750 kDa, bead diameter: 60-140 μm). The introduced toxin solution displaces the buffer solution in the gel when it penetrates the gel. After complete penetration of the venom solution into the gel, 165 mL of solvent (0.25 M Tris/HCl, pH 6.5-7.3, 1.92 M glycine) is added to the column in portions (without allowing the gel to run dry). This solvent displaces the poison solution as it passes through the gel. The first 15 mL that drip out of the bottom of the column are residual gel buffer and are discarded. Subsequently, 40 fractions of 4 mL are collected. The chemical and physical properties of this separation system resulted in the 4 mL for the individual fractions. SDS-PAGE is used to check that only one component is really found per fraction. Only one band per fraction.

For SDS-PAGE, Roti Load 1+2 (Carl Roth GmbH & Co KG, Karlsruhe: SDS, glycerol, bromophenol blue, phosphate buffer, Roti Load 1 with mercaptoethanol, Roti Load 2 without mercaptoethanol) is used as an application buffer for the fraction on the gel for peptide binding and protein protection.

The individual fractions after column separation are collected separately in clean, previously sterilized and screwable Teflon vials.

### Example 3: Production of Urticaceae Peptide Toxins

The leaves of the Urticaceae species are extracted as described in more detail above in order to obtain the total toxin cocktail. The total toxin is then obtained by column chromatography, for example by HPLC. Fractions 2, 7, 8 and 11 contain the brain tumor-destroying substances in the Dendrocnide species Dendrocnide moroides and Dendrocnide excelsa. Fractions 5 and 7 contain the brain tumor-destroying substances in the Urera species Urera baccifera and Urera nitida.

Using SDS-PAG electrophoresis with Coomassie blue staining, the following (averaged) molecular weights of the freeze-dried substances were obtained:

Dendrocnide moroides and Dendrocnide excelsa:

| | |
|---|---|
| Fraction 2 | 35 000 Da |
| Fraction 7 | 40 000 Da |
| Fraction 8 | 52 000 Da |
| Group 11 | 75 000 Da |

Urera baccifera and Urera nitida:

| | |
|---|---|
| Fraction 5 | 35 000 Da |
| Fraction 7 | 51 000 Da |

Fraction 4 corresponds to HT1 peptide toxin, fraction 10 to HT2 peptide toxin.

Like HT1 and HT2, the peptide toxins from Girardinia and Nanocnide were tested on commercially available standard glioblastoma and astrocytoma cell cultures and also showed the desired effect.

According to the invention, a kit of parts can be present as a kit, a device and/or a system arrangement.

1. A pharmaceutical composition for the treatment of a brain tumor comprising in a pharmaceutically effective amount a) peptide toxin from plants of the genus Dendrocnide, plants of the genus Urera, plants of the genus Nanocnide, plants of the genus Girardinia; and b) total toxin or peptide toxins from plants of the genus Laportea having a molecular weight of 15 kDa to 100 kDa and the ability to cross the blood-brain barrier.

2. The pharmaceutical composition according to claim 1, characterized in that it additionally contains inactivated Parapoxvirus ovis, preferably strain D1701.

3. The pharmaceutical composition according to claim 1, characterized in that the plants of the genus Dendrocnide are selected from the group consisting of the species Dendrocnide corallodesme and/or Dendrocnide cordata and/or Dendrocnide cordifolia and/or Dendrocnide excelsa and/or Dendrocnide gigantea and/or Dendrocnide meyeniana and/or Dendrocnide moroides and/or Dendrocnide peltata and/or Dendrocnide sinuata and the plants of the genus Urera selected from the group consisting of the species Urera caracasana and/or Urera baccifera and/or Urera expansa and/or Urera kaalae and/or Urera nitida and/or Urera simplex and the plants of the genus Nanocnide selected from the group consisting of the species Nanocnide japonica and/or Nanocnide lobata and/or Nanocnide closii and the plants of the genus Girardinia selected from the group consisting of the species Girardinia bullosa and/or Girardinia diversifolia, the species Dendrocnide moroides, Dendrocnide excelsa, Urera baccifera, Urera kaalae, Urera nitida and Girardinia diversifolia being particularly preferred.

4. The pharmaceutical composition according to claim 1, characterized in that the peptide oxide from plants of the genus Dendrocnide is the HTDen1 and/or the HTDen2 peptide toxin.

5. The pharmaceutical composition according to claim 1, characterized in that it additionally contains Lachesis D6.

6. The pharmaceutical composition according to claim 1, characterized in that the plants of the genus Laportea are selected from the group consisting of the species Laportea bulbifera, Laportea canadensis, Laportea cuspidata, Laportea grossa, Laportea interrupta, Laportea mooreana, Laportea ruderalis, Laportea septentrionalis.

7. The pharmaceutical composition according to claim 1, characterized in that the plants of the genus Laportea are preferably the species Laportea bulbifera and/or Laportea canadensis and/or Laportea ruderalis.

8. The pharmaceutical composition according to claim 1, characterized in that it further comprises peptide toxins of dendrocnide, urera, nanocnide and/or girardinia.

9. A composition according to claim 1 for use in a method of treating brain tumors and glioblastomas.

10. A method of preparing a composition according to claim 1, comprising the steps of: —Providing an isotonic sodium chloride solution with dendrocnide peptide toxin and/or urera peptide toxin and/or nanocnide peptide toxin and/or Girardinia peptide toxin; optionally adding a further dendrocnide peptide toxin and/or urera peptide toxin, and/or nanocnide peptide toxin and/or Girardinia peptide toxin to the sodium chloride solution; and adding a total toxin/extract or peptide toxins from plants of the genus Laportea, having a molecular weight of 15 kDa to 100 kDa and the ability to penetrate the blood-brain barrier; mixing the ingredients, in particular by shaking the mixture.

11. The method according to claim 10, wherein an immunomodulator, preferably inactivated parapoxvirus ovis, is additionally added before shaking.

12. The method according to claim 10, wherein at least one of the dendrocnide peptide toxins and/or the urera peptide toxins and/or the nanocnide peptide toxins and/or the girardinia peptide toxins are dissolved in Lachesis D6.

13. The method according to claim 10, wherein the first dendrocnide peptide toxin is a dendrocnide peptide toxin HTDen1.

14. The method according to claim 10, wherein the second dendrocnide peptide toxin is a dendrocnide peptide toxin HTDen2.

15. A kit of parts for the treatment of brain tumors and glioblastomas comprising i) a pharmaceutical composition according to claim 1; and ii) extract of frankincense and/or bamboo.
